(19) United States
(12) Patent Application Publication
(10) Pub. No.: US 2013/0071808 A1
(43) Pub. Date: Mar. 21, 2013

(54) System, Device and Method for Determining Dental Erosion

(71) Applicant: Elisabeth Wilhelmina Hendrika Van der Laan, Amsterdam (NL)

(72) Inventor: Elisabeth Wilhelmina Hendrika Van der Laan, Amsterdam (NL)

(21) Appl. No.: 13/520,465

(22) PCT Filed: Jan. 4, 2011
(86) PCT No.: PCT/NL11/50001

§ 371 Date: Sep. 21, 2012

(30) Foreign Application Priority Data
Jan. 4, 2010 (NL) 2004046
Jan. 27, 2010 (NL) 2004154

(51) Int. Cl. A61B 6/14

(52) U.S. Cl. CPC A61B 6/145
USPC 433/29; 433/215

(57) ABSTRACT

A system for determining dental erosion by measuring the translucency of a tooth at the same location of the tooth at least two points in time during the lifetime of a person or an animal, said system comprising a device provided with a carrier on which a light source and a light sensor are mounted, which carrier can be placed against a tooth or a molar, preferably against the cutting edge thereof, and which system further comprises electronic processing means, which are designed to convert the signal from the light sensor into a quantitative value, means which are designed to store a first determined value from the sensor, means which are designed to compare a second determined value from the sensor with the first determined value, and means which are designed to communicate a representative of the result of the comparison to a user.

The present invention relates to a system for determining dental erosion. Such systems are used in dental practice, for example. The term “dental erosion” as used herein is understood to means any form of decrease of the thickness of a tooth or a molar.

The object of the invention is in general to provide means by which dental erosion can be determined in a better and simpler manner so as to improve the treatment or the recovery of a person's teeth.

The object of the invention is to provide a precise determination of the degree of or the detection of the presence of erosion, so that timely measures can be taken to detect any dental erosion and dental wear, for example caused by loss of enamel, that may already have set in and stop such erosion and wear. Erosion is mainly caused by the consumption of acidic foodstuffs.

In order to accomplish that object, the device comprises a carrier on which a light source and a light sensor are mounted, which carrier can be placed against a tooth or a molar, preferably against the cutting edge thereof, wherein the system further comprises electronic processing means, which are designed to convert the signal from the light sensor into a quantitative value, means which are designed to store a first determined value from the sensor, means which are designed to compare a second determined value from the sensor with the first determined value, and means which are designed to communicate a representative of the result of the comparison to a user. From the signal that is delivered by the light sensor, the translucency, i.e. the transparency or opaqueness, of the tooth or the molar can be determined.

The translucency, degree of transparency (or opaqueness) of a tooth or molar is a measure of the thickness of the tooth at that location. By measuring the translucency or degree of transparency at a first moment, it is possible to measure the condition and the thickness of the tooth or the molar, or to determine whether there is dental erosion. By subsequently measuring the translucency or degree of transparency at the same location on the tooth or the molar once again at one or more points in time, it is possible to monitor the development of dental erosion and to determine a treatment plan, if necessary.

In a preferred embodiment, the light source and the light sensor are directed towards each other, such that the carrier can be placed against a tooth or a molar whilst the tooth or the molar is present between the light source and the light sensor. In another preferred embodiment, the light source and the light sensor may be disposed in substantially the same direction on the carrier, such that when the carrier is placed against the tooth or the molar, the light emitted by the light source that is reflected in the tooth or the molar is received by the sensor.

The carrier. is preferably provided with a linear stop, which stop extends parallel to and spaced from the axis between the light source and the light sensor, such that the stop can be placed against the side of the tooth or the molar when the tooth or the molar is present between the light source and the light sensor. The linear stop is preferably formed by an edge of a plate which extends from the carrier. In this way the location of the measurement can be precisely set. Preferably, the device is further provided with adjustable screening means for preventing the incidence of external light on the light sensor, which screening means can be set in dependence on the shape of the tooth or the molar. Preferably, the screening means are formed by a flexible material which adapts its shape to that of the tooth, the molar or other part of the body.

In a first preferred embodiment, the carrier is provided with a handgrip to be engaged by a user. In a second embodiment, the carrier is a unit which can be placed in the mouth of a person or an animal.

The light source preferably comprises a light emitting diode, preferably a white LED, and the light sensor preferably comprises a light dependent resistor, a photodiode, a photo transistor or a camera, preferably a photo camera, a 2D camera, a 3D camera or a web camera, preferably a CCD sensor.

The electronic processing means preferably comprise a display. The device preferably comprises a pushbutton, a pressure switch, a slide knob or rotary knob for activating the device, and preferably also for activating the photo camera. With such a pushbutton, a light pulse of a specific duration can be generated, for example. Preferably, is also possible to set the intensity or the power of the light source by means of the pushbutton, the pressure switch, the slide knob or the rotary knob. In a preferred embodiment, the device comprises a switch which is designed to activate the light source and/or the light sensor for a predetermined or an adjustable period of time. Preferably, the intensity, the sensitivity and/or the power of the light source and/or the sensor can be set, preferably by means of the pushbutton, the pressure switch, the slide knob or the rotary knob.

In a first preferred embodiment, the electronic processing means preferably comprise a connection for a data transfer cable, which is preferably also the power source, such as a USB connection or a FireWire connection. In another preferred embodiment, the power source comprises a rechargeable battery. In yet another preferred embodiment, the power source comprises an electric power plug.

The invention also relates to a device for determining dental erosion by measuring the translucency of a tooth at the same location of the tooth at at least two points in time during the lifetime of a person or an animal, said device comprising a carrier, on which a light source and a light sensor are mounted, which carrier can be placed against a tooth or a molar, wherein the device further comprises electronic processing means, which are designed to convert the signal from the light sensor into a quantitative value, means which are designed to store a first determined value of the sensor, means which are designed to compare a second determined value of the sensor with the first determined value, wherein the electronic processing means comprise a display on which the quantitative value is displayed.

The invention also relates to a method for determining dental erosion, wherein the translucency or the degree of transparency of a tooth or a molar is measured at the same location of the tooth or the molar at at least two points in time during the lifetime of a person or an animal.

In a preferred embodiment, the sensor is a camera, by means of which a photo is taken, and the colour and/or the brightness of the photo is analysed.

The invention will now be explained in more detail with reference to embodiments shown in the figures, in which:

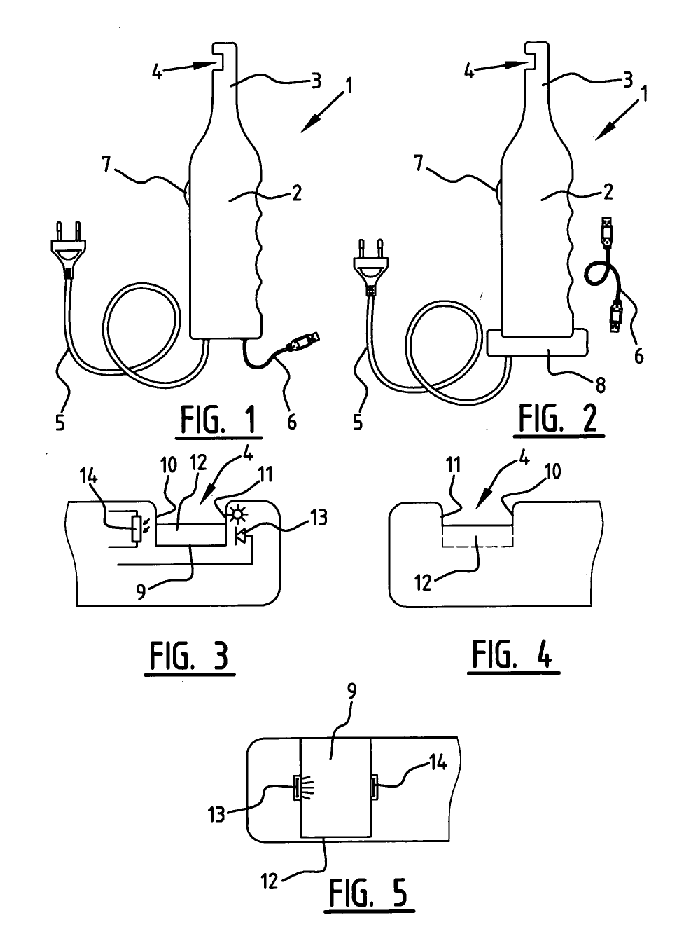

FIG. 1 is a front view of a first embodiment of a device according to the invention;

FIG. 2 is a front view of a second embodiment of the device according to the invention;

FIG. 3 is a schematic front view of a part of the devices of FIGS. 1 and 2;

FIG. 4 is a schematic rear view of a part of the devices of FIGS. 1 and 2;

FIG. 5 is a schematic top plan view of a part of the devices of FIGS. 1 and 2;

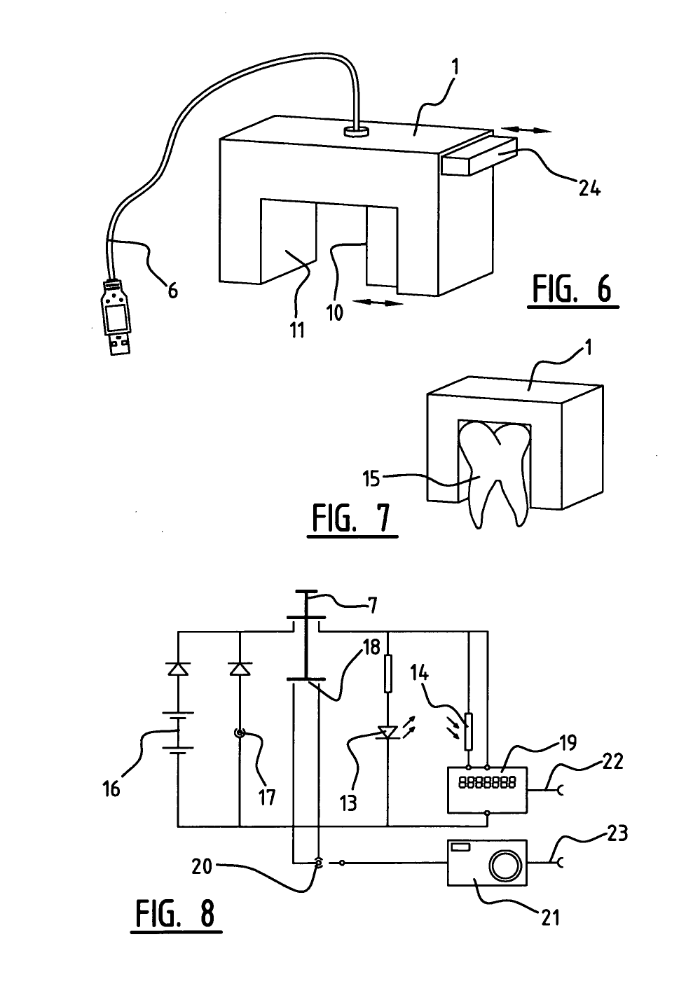

FIG. 6 is a perspective front view of a third embodiment of the device according to the invention;

FIG. 7 is a schematic perspective view of the device according to the invention placed over a molar; and

FIG. 8 is an electric diagram of the device according to the invention.

The device according to FIG. 1 comprises a carrier 1 provided with a handgrip 2 and a handle-shaped end 3. A recess 4 is formed in the handle-shaped end 3, which recess can partially accommodate a person's tooth. An electric lead 5 and a USB data cable 6 are connected to the carrier 1. The handgrip 2 is provided with a control button 7.

In the embodiment shown in FIG. 2, the handgrip 2 of the carrier at 1 is placed in a dock 8, to which the electric lead 5 is connected. The handgrip accommodates a rechargeable battery. The battery can be charged by cooperating connectors in the dock 8 and the handgrip 2. In this embodiment, the USB data cable 6 can be disconnected from the handgrip 2 or from the dock 8.

According to FIGS. 3, 4 and 5, the recess 4 in the handle-shaped end 3 is substantially a U-shaped channel which extends from one side of the carrier 1 to the other side of the carrier 1 and which comprises a bottom 9, an outer wall 10 and an inner wall 11. On one side, the recess 4 is partially closed by an upright wall 12, which forms a stop that can be placed against the side of a tooth. Not shown in the figures are flexible or non-flexible closures, for example made of rubber, of the recess 4 on both sides, which closures can be depressed by a tooth or a molar and which are intended to keep out external light and/or fluids, such as saliva or blood, during measurements.

The handle-shaped end is provided with a light source 13 and a light sensor 14. In this example, the light source 13, for example an LED, a laser source or other lamp, is disposed in a recess in the inner wall 10, such that the light source shines on the outer wall 11 in use. Disposed in a recess in the outer wall 11 is the light sensor 14.

In an alternative embodiment (not shown), the handle-shaped end 3 provided with the light source 13 and the light sensor 14 extends transversely to the handgrip 2, so that it is possible, if desired, to carry out a measurement with the light source placed against the front side of the tooth and the sensor placed against the rear side of the tooth, or conversely. In yet another embodiment, the handle-shaped end 3 is connected to the handgrip 2 by means of a hinge or via a flexible art. In yet another embodiment, the light source 13 and the light sensor 14 are disposed on the handle-shaped end, facing in the same direction, preferably radially away from the axis of the device, so that the handle-shaped end can be placed against the outer side of a tooth or a molar, as a result of which the light source 13 will shine into the tooth or the molar and the sensor 14 will measure the light reflected from said tooth or molar.

According to the embodiment shown in FIG. 6, the carrier 1 comprises a block-shaped element provided with a substantially U-shaped channel, which channel extends from one side of the carrier 1 to the other side of the carrier 3. and which is provided with a bottom 9, an inner wall 10 and an outer wall 11. The carrier 1 is provided with a light source 13 and a light sensor 14. The light source 13 is disposed in a recess in the inner wall 10, such that the light source shines on the outer wall 11 in use. Disposed in a recess in the outer wall 11 is the light sensor 14. The inner wall 10 is mounted for sliding movement in its normal direction in the carrier and is connected to a pushbutton 24, such that the inner wall 10 can be slid in the direction of the outer wall 11 after the carrier has been placed on a molar, until the two walls abut against the molar. The light source 13 and the light sensor 14 can be connected to a computer or other control and data processing device by means of a USB data and power cable 6.

According to embodiments not shown in the figures, the carrier 1 is adapted for being mounted to another device, for example an (electrical) toothbrush, a thermometer, a dental curing lamp (for composite fillings and sealings, among other things), a plaque score meter, a caries detector lamp, a digital printing device, a CAD/CAM device, a colour, shape or thickness measuring device, a dentist's chair or treatment unit.

FIG. 7 shows the carrier 1 placed on a molar 15, with the inner wall 10 abutting against the inner side of the molar and the outer wall 11 abutting against the outer side of the molar.

According to FIG. 8, the device comprises a rechargeable battery 16 and an adapter/charger connection 17, which are connected to the LED light source 13 and the LDR light sensors 14 by means of a switch 18. The switch 18 can be operated by means of the control button 7. The control button 7 may be a pushbutton, which puts the switch in the on-position for as long as the button is pressed, and which puts the switch in the off-position when the button is released. The control button 7 may also be an on/off button, which puts the switch permanently in the on-position or the off-position, as desired. The switch 18 may be designed such that a pulse timer will deliver a pulse of a predetermined or set duration and intensity upon operation of the control button 7. The pulse time may be variable, for example between 0.000001 sec and 1 hour, but preferably the pulse time is or can be set between 0.00001 and 60 seconds, more preferably between 0.01 and 10 seconds, even more preferably between 0.5 and 5 seconds.

The light source 14 is furthermore connected to an electronic display screen 19 in the carrier 1, on which the strength of the signal from the light sensor 14 is displayed. In this way it is possible to measure and register the degree of transparency, for example, which, seen in the course of time, is a representative of the degree of the erosion of the tooth or the molar at that location.

Since the light source 13 may become weaker with the passage of time or exhibit other variations in light intensity, the system can be calibrated. By making a calibration measurement prior to a measurement of the transparency of a tooth or molar, with no tooth or molar disposed in the light path between the light source 13 and the light sensor 14, it is possible to determine the maximum transparency value, which measure can be used for correcting subsequently measured values.

In an alternative embodiment (not shown), the light source 13 and the light sensor have relatively large effective transmission and receipt areas, and a, possibly detachable, screen provided with a diaphragm (opening) can be placed in front of the light source 13 and/in front of the light sensor 14 at a particular location, so that the transparency at that particular location can be measured. In another embodiment (not shown), the light source 13 and/or the light source 14 may to that end also be movable, in which case they will have a small effective area. The two embodiments may also be combined. The diaphragm may be changeable as regards size and shape, and be movable.

Using the light source 13 and the sensor 14, the various values of transparency of the entire tooth or molar (or other part of the body), or a part thereof, can be determined at different locations. This may be done by, for example, x/y measurement, location, or by a pre-set interval of location measurement (from bottom to top, from top to bottom, from left to right, from right to left, or diagonally). The result of the measurements is then shown in quantitative measured values.

The light sensor is further connected to a USB connection 22. By means of said connection the device can be connected to a computer for further data processing and data storage. The computer may for example be provided with a programme for converting the measured light values into image (shades of grey or other colours) or into data.

The switch 18 is further connected to a potential-free contact 20 in the carrier 1, to which a camera 21 can be connected. In this way shots of the tooth or molar can be taken during the period that the light source 13 is on. The camera 21 may be a photo camera, a 2D camera, a 3D camera, a video camera or a web camera, for example, and be connected to, for example, a USB cable 23 or to a computer for viewing the recorded images. In this way it is possible, for example, to detect caries, erosion or other irregularities, or to determine the colour and transparency of a tooth or a molar.

1. A system for determining dental erosion by measuring the translucency of a tooth at the same location of the tooth at at least two points in time during the lifetime of a person or an animal, said system comprising a device provided with a carrier on which a light source and a light sensor are mounted, which carrier can be placed against a tooth or a molar, preferably against the cutting edge thereof, and which system further comprises:
electronic processing means for converting the signal from the light sensor into a quantitative value;
means for storing a first determined value from the sensor;
means for comparing a second determined value from the sensor with the first determined value; and
means for communicating a representative of the result of the comparison to a user.

2. A system according to claim 1, wherein the light source and the light sensor are directed towards each other, such that the carrier can be placed against a tooth or a molar whilst the tooth or the molar is present between the light source and the light sensor.

3. A system according to claim 2, wherein the carrier is provided with a linear stop, which stop extends parallel to and spaced from the axis between the light source and the light sensor, such that the stop can be placed against the side of the tooth or the molar when the tooth or the molar is present between the light source and the light sensor.

4. A system according to claim 3, wherein the linear stop is formed by an edge of a plate which extends from the carrier.

5. A system according to claim 1-4, wherein the carrier is provided with a handgrip to be engaged by a user.

6. A system according to claim 1-4, wherein the carrier is a unit which can be placed in the mouth of a person or an animal.

7. A system according to claim 1, wherein the light source comprises a light emitting diode or a laser source.

8. A system according to claim 1, wherein the light sensor comprises a light dependent resistor, a photodiode, a photo transistor, a 2D camera, a 3D camera, a web camera, a photo camera, a CCD sensor or another camera.

9. A system according to claim 1, wherein the electronic processing means for converting the signal from the light sensor into a quantitative value comprises a display.

10. A system according to claim 1, wherein the device comprises a pushbutton, a pressure switch, a slide knob or rotary knob for activating the device.

11. A system according to claim 1, wherein the device comprises a switch which is designed to activate the light source and/or the light sensor for a predetermined or an adjustable period of time, for example in the form of a pulse.

12. A system according to claim 1, wherein the electronic processing means for converting the signal from the light sensor into a quantitative value comprises a connection for a data transfer cable, which is preferably also the power source, such as a USB connection or a FireWire connection.

13. A system according to claim 1, wherein the means for storing comprise an electronic storage means, such as a memory chip, magnetic storage means or optical storage means.

14. A system according to claim 1, wherein the means for comparing comprise an electronic processing means and a software program to be loaded into said electronic processing means.

15. A system according to claim 1, wherein the light source and the light sensor are disposed in substantially the same direction on the carrier, such that when the carrier is placed against the tooth or the molar, the light emitted by the light source that is reflected in the tooth or the molar is received by the sensor.

16. A device for determining dental erosion by measuring the translucency of a tooth at the same location of the tooth at at least two points in time during the lifetime of a person or an animal, said device comprising a carrier, on which a light source and a light sensor are mounted, which carrier can be placed against a tooth or a molar, wherein the device further comprises:
electronic processing means for converting the signal from the light sensor into a quantitative value;
means for storing a first determined value of the sensor; and
means for comparing a second determined value of the sensor with the first determined value,
wherein the electronic processing means for converting the signal from the light sensor into a quantitative value comprise a display on which the quantitative value is displayed.

17. A device according to claim 16, wherein the light source and the light sensor are directed towards each other, such that the carrier can be placed against a tooth or a molar whilst the tooth or the molar is present between the light source and the light sensor.

18. A method for determining dental erosion comprising:
measuring the translucency or the degree of transparency of a tooth or a molar at the same location of the tooth or the molar at at least two points in time during the lifetime of a person or an animal.
